(19) United States
(12) Patent Application Publication
(10) Pub. No.: US 2012/0244199 A1
(43) Pub. Date: Sep. 27, 2012

(54) Skin Nourishing Ball

(76) Inventor: Sheng-Jen Yeh, Kaohsiung City (TW)

(21) Appl. No.: 13/052,541

(22) Filed: Mar. 21, 2011

(51) Int. Cl. A61K 8/11; A61Q 19/00; A61K 8/98

(52) U.S. Cl. 424/401; 424/582

(57) ABSTRACT

A skin nourishing ball is disclosed. The skin nourishing ball has as a transparent ball body that includes a casing made of fish-scale collagen and a fish embryo extract particle enclosed in the casing. The fish embryo extract is taken from a fertilized fish embryo and therefore highly stable against deterioration. In addition, the fish embryo extract is effective in reducing fine lines on the skin, improving skin elasticity, and enhancing the spontaneous moisture-retaining ability of skin. Due to the granular configuration of the skin nourishing ball, it is possible not only to take a specific quantity of such balls for use, but also to control the quantity with ease, thereby preventing the user from applying an excessive or insufficient quantity of the skin nourishing balls to the skin.

### BACKGROUND OF THE INVENTION

1. Technical Field

The present invention relates to a nourishing ball that is applicable to the human skin. More particularly, the present invention relates to a skin nourishing ball whose granular shape makes it possible to apply a specific quantity of such balls to the skin for nourishment and thereby prevent excessive or insufficient use of the skin nourishing balls.

2. Description of Related Art

Generally speaking, lack of collagen results in aging, inelastic, wrinkled, or loose skin. Hence, it is not uncommon nowadays to add collagen, L-ascorbic acid, growth factors, biochemically active peptides, or like ingredients into cosmetic or skin care products to increase the smoothness, firmness, and moisture-retaining ability of skin. The aforementioned ingredients are effective in reducing fine lines or wrinkles, enhancing skin elasticity, and improving the spontaneous moisture-retaining ability of skin.

According to user feedbacks, commercially available collagen or collagen-added skin care products typically take the form of clear liquid or emulsion and are very expensive because they are packaged in small “ampoules” and seem to have high purity.

However, due to their clear liquid or emulsion form, collagen or collagen-added skin care products on the market must be poured or squeezed out for use. As a result, it is difficult to control the amount for each use, and any excessive amount that has been poured or squeezed out becomes a waste, which is very inconvenient. Moreover, the skin care products, once opened for use, are exposed to air. The exposure will cause the ingredients and tissues in the unused portions of the products to gradually deteriorate; in other words, the conventional products have poor storability.

### BRIEF SUMMARY OF THE INVENTION

In view of the aforesaid drawbacks of the existing skin nourishing products in use and storage, the present invention discloses a skin nourishing ball having a transparent ball body. The ball body includes a casing made of fish-scale collagen and a fish embryo extract particle enclosed in the casing. The fish embryo extract particle further contains some shiny powder.

Preferably, the fish embryo extract particle is made of a grouper embryo.

Preferably, the grouper embryo is fertilized.

Preferably, the shiny powder is gold powder.

Preferably, the casing is coated with a film.

The present invention has the following advantages:

1. By shaping the fish-scale collagen-based casing and the fish embryo extract particle into a granular skin nourishing ball, users are allowed to take a specified quantity of such balls for use and easily control the quantity taken. Thus, excessive or insufficient application of the skin nourishing balls can be avoided.

2. With the skin nourishing ball being elastic, and the fish embryo extract particle containing shiny powder, a plurality of such balls can be easily and unambiguously distinguished from one another during use.

3. The skin nourishing ball can be mixed with a medium or an intensive nourishing fluid and stored therewith in a bottle so as to keep the moistness and activity of the skin nourishing ball. In addition, the skin nourishing ball will not break until it is rubbed against the skin, and once the ball is broken, the ingredients thereof are released and can be readily absorbed by the skin.

4. As the fish-scale collagen-based casing is coated with a soft, translucent, and elastic film to isolate the skin nourishing ball against oxidation, the ingredients and tissues of both the casing and the fish embryo extract particle are protected from damage, thus allowing the skin nourishing ball to be stored without its quality being compromised.

### BRIEF DESCRIPTION OF THE SEVERAL VIEWS OF THE DRAWINGS

A detailed description of further features and advantages of the present invention is given below with reference to the accompanying drawings, in which:

FIG. 1 is a sectional view of the structure of the present invention;

FIG. 2 is a sectional view of an embodiment of the present invention that contains shiny powder;

FIG. 3 is a sectional view of another embodiment of the present invention;

FIG. 4 schematically shows the present invention being mixed with a medium and stored in a container; and

FIG. 5 schematically shows how the present invention is used on the skin.

### DETAILED DESCRIPTION OF THE INVENTION

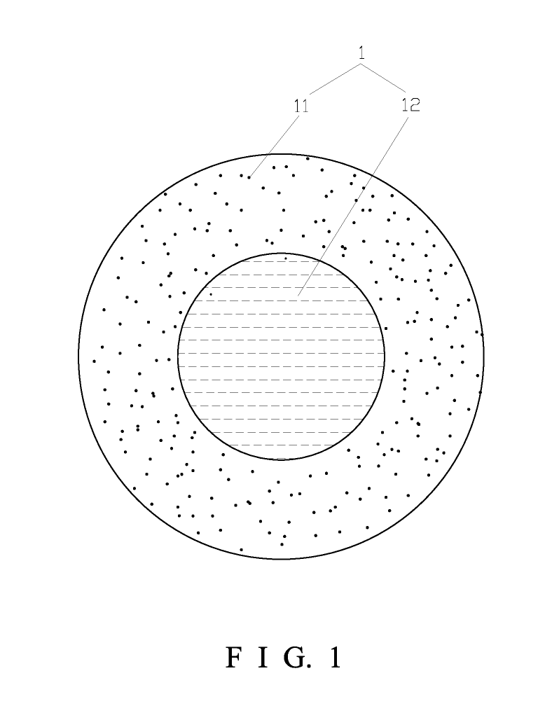

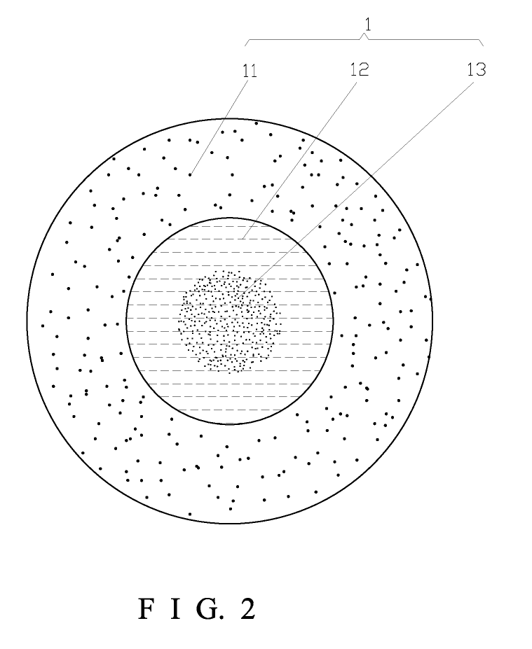

Referring to FIG. 1 and FIG. 2, the present invention provides a skin nourishing ball having a transparent ball body 1. The ball body 1 includes a casing 11 made of fish-scale collagen. Enclosed in the casing 11 is a fish embryo extract particle 12. The fish embryo extract particle 12 is composed of the ingredients of a fertilized grouper embryo and, thanks to the fertilized state of the embryo, is highly stable against deterioration. The fish-scale collagen-based casing 11 and the fish embryo extract particle 12 can promote the growth of skin cells, as well as the excretion of collagen and extracellular matrix, and are therefore effective in reducing fine lines or wrinkles on the skin, increasing skin elasticity, and improving the spontaneous moisture-retaining ability of skin. Further, as the ball body 1 has a granular configuration, it is possible to take a specific quantity of the disclosed skin nourishing balls for use and easily control the quantity taken. As shown in FIG. 2, the fish embryo extract particle 12 in the ball body 1 is mixed with some shiny powder 13, such as gold powder, to make the ball body 1 conspicuous. By so doing, a plurality of ball bodies 1 can be readily distinguished from one another to facilitate control of the quantity taken for each use. Hence, not only can the skin nourishing balls be applied to the user's skin in the proper quantity for skin nourishment to prevent wasteful or inadequate use, but also the shiny powder 13 produces a flickering effect on the skin.

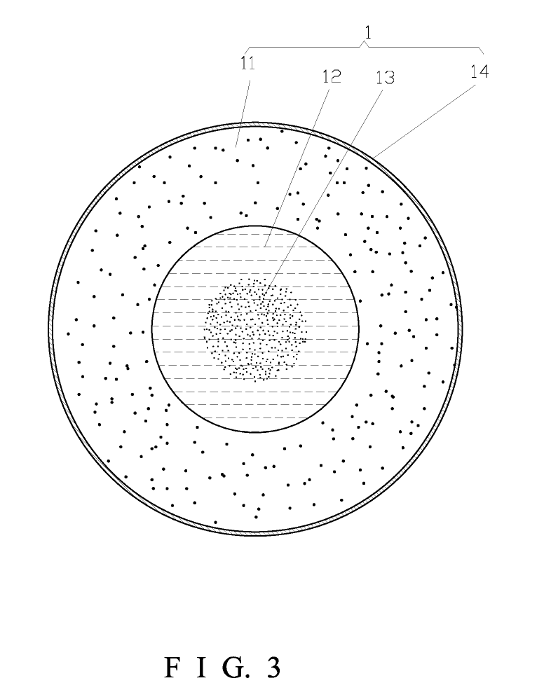

In another embodiment of the present invention as shown in FIG. 3, a translucent elastic film 14 is coated on the surface of the fish-scale collagen-based casing 11 of the ball body 1. The film 14, which is intended for isolation and anti-oxidation, allows the transparent fish-scale collagen-based casing 11 and the fish embryo extract particle 12 to maintain their original ingredients and avoid tissue damage. In other words, the disclosed skin nourishing ball has good storability.

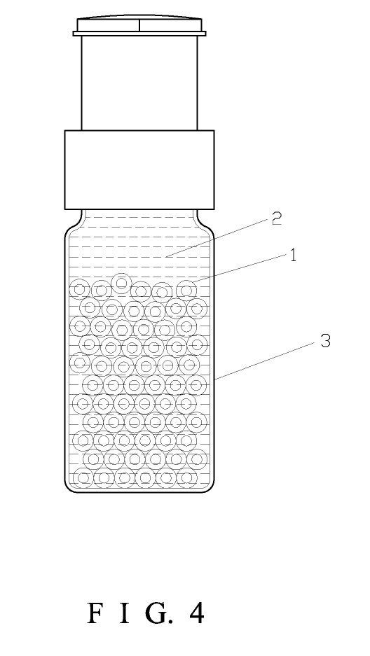

In addition, the skin nourishing ball of the present invention can be kept in a container. Referring to FIG. 4, a plurality of ball bodies 1 are mixed with a medium 2 in advance, wherein the medium 2 is an intensive nourishing fluid or water. Then, the mixture is put into a container 3, such as an ampoule. The medium 2 serves to keep the ball bodies 1 moist and active so that both use and storage of the skin nourishing balls are made easy.

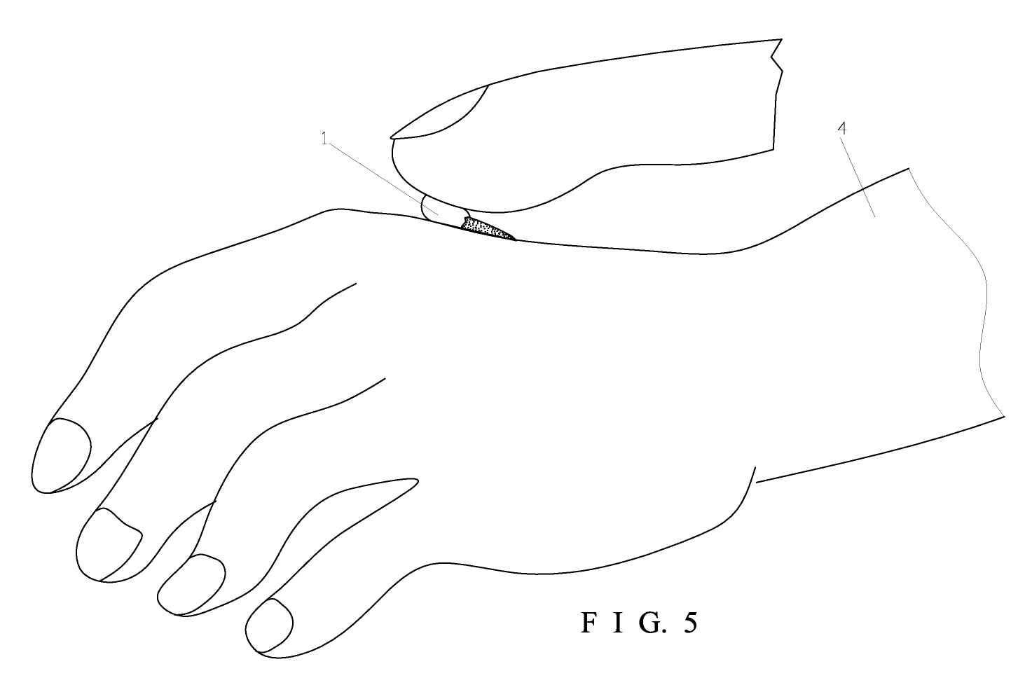

Application of the disclosed skin nourishing ball to the user's skin is now described with reference to FIG. 5. A specific quantity of ball bodies 1 are taken out of the container 3 and placed on the skin 4. Then, the ball bodies 1 are pushed and rubbed against the skin 4 such that the ingredients of the fish-scale collagen-based casings 11 and of the fish embryo extract particles 12 are released from the ball bodies 1, to be absorbed by the skin 4 for nourishing and/or cosmetic purposes.

What is claimed is:

1. A skin nourishing ball, having a transparent ball body, the ball body comprising a casing and a fish embryo extract particle enclosed in the casing, wherein the casing is made of fish-scale collagen, and the fish embryo extract particle contains shiny powder.

2. The skin nourishing ball of claim 1, wherein the fish embryo extract particle is made of a grouper embryo.

3. The skin nourishing ball of claim 2, wherein the grouper embryo is fertilized.

4. The skin nourishing ball of claim 1, wherein the shiny powder is gold powder.

5. The skin nourishing ball of claim 1, wherein the casing is coated with a film.
